(19) United States
(12) Patent Application Publication
(10) Pub. No.: US 2007/0078292 A1
(43) Pub. Date: Apr. 5, 2007

(54) Electromagnetic fields for systemic effect in therapy

(75) Inventors: Marko S. Markov, Williamsville, NY (US); Carlton F. Hazlewood, The Woodlands, TX (US); Arthur D. Ericsson, Houston, TX (US); Richard Bailey, Houston, TX (US)

Correspondence Address: ALBERT B. KIMBALL, JR.;BRACEWELL & PATTERSON L L P, 711 LOUISANA SUITE 2300, HOUSTON, TX, 77002, US

(73) Assignee: Electromagnetic Resources, Inc., Houston, TX (US)

(21) Appl. No.: 11/243,896

(22) Filed: Oct. 5, 2005

(51) Int. Cl. A61N 2/00; A61B 17/52

(52) U.S. Cl. 600/9

(57) ABSTRACT

A new mechanism of therapeutic action of electromagnetic fields, based upon systemic effect, applies selected EMF signals to cause efficacious therapeutic effects at sites distant from the point of application. The method of generating specific EMF waveshape and application of this exogeneous signal for therapeutic purposes allows the benefit to be achieved via the systemic effect. The EMF signals take the form of a repetitive sequence of semi sinewaves, resulting in a pulsating waveform occurring at a frequency in the range of from about 50 to about 300 pulses per second. Bridge rectification is applied to a sinusoidal signal, and a resultant therapy signal takes the form of a repetitive sequence of semi sinewaves of up to about 300 pps with a periodically recurring DC component. The shape of alternating pulses in the resultant therapy signal is modified under computer control to achieve therapeutic effects. Flux densities in the range of several hundred Gauss up to several thousand Gauss are generated. The treatment with the resultant therapy signal may be applied to an arm or leg of a therapy recipient for a sufficient length of time to cause the recipient's entire blood volume to be exposed to the therapeutic field such that this can initiate effects in cells, tissues, and organs distant from the point of application

### BACKGROUND OF THE INVENTION

1. Field of the Invention

The present invention relates to electromagnetic field therapy, and particularly to the way selected magnetic or electromagnetic fields are generated and applied for treatment of selected various diseases, pathologies and injuries. Healing effects are obtained according to the present invention as a result of a systemic effect, i.e. the applied therapy having beneficial effects at sites distant from the site of therapeutic application.

2. Description of the Related Art

Within the last two decades, there has been an increasing interest on the part of the general public toward the use of magnetic fields for pain control. It would also be fair to say that there has been increasing interest in the therapeutic use of magnetic fields, stimulated in large part by recent advances in alternative and complementary medicine.

During the past 25 years more than two million patients have been treated worldwide for a large variety of injuries, pathologies and diseases. This large number of patients exhibited a success rate of approximately 80%, with virtually no reported complications.

At present, electromagnetic therapeutic modalities can be categorized into six groups: permanent magnetic fields, low frequency sine waves EMF, pulsed low frequency electromagnetic fields (PEMF), pulsed radiofrequency fields (PRF), millimeter waves and transcranial magnetic stimulation.

Before discussing the specifics of the choice of magnetic field modality, the necessity should be emphasized to evaluate the location of the clinical target and the magnetic field a target for treatment has needed to receive. It should be taken into account that different types of magnetic field have different characteristics, and they may even have variations in the field characteristics at the target site.

The contemporary magnetotherapy originated in Japan immediately after World War II and later attracted the interest of physicians in Asia (China and India) and Europe (mainly Romania and the former Soviet Union). During the period 1960-1985 a number of European countries manufactured and distributed some form of locally developed or originated magnetotherapeutical system. So far as is known, the first clinical application of electromagnetic stimulation in the United States was in 1974, and that method was approved for treatment of non-union and delayed fractures.

The first contemporary book of which Applicants are aware on magnetotherapy was written by N. Todorov and published in 1982 in Bulgaria. The book summarized experience with treatment of some 2700 patients with 33 different pathologies. Numerous publications over the past 25 years have suggested that exogenous magnetic and electromagnetic fields might have effects on a large number of biological processes, many of which are important for therapy.

A recent book, BIOELECTROMAGNETIC MEDICINE, of which one of Applicants was co-editor and which was published in April, 2004 covered numerous applications of electromagnetic field (or EMF) therapy from fracture repair and wound healing to epilepsy, Alzheimer's and Parkinson's diseases. That recent book also reviewed the clinical use of EMF for treatment of cancer.

Magnetic and electromagnetic fields have been used for treatment of various musculoskeletal injuries and pathologies which occurred due to a variety of causes: injury, over-use of a particular body part, or the effects of illness or infection. In the United States the most effective use of magnetic fields (or MF) was reported for fracture and wound management, reduction of pain and inflammation.

It is now well accepted that the EMF can provide a practical, exogenous method for inducing cell and tissue modifications, which correct selected pathological states. It should be noted that very little is known about the mechanisms of action and this limitation has seriously restricted the application of magnetic and electromagnetic fields in clinical practice in the United States. More research and publications can be found in European literature—more than 2,000 papers on the beneficial effects of magnetic and electromagnetic stimulation can be found. Most of these papers, however, present results of open studies, and only a few have been done as double blind, controlled studies.

There is a large body of experimental and clinical data suggesting that various exogenous MF at surprisingly low levels can affect a large variety of tissues and processes, most of which are of critical importance for diagnostics and therapy. The longest clinical applications of magnetic fields in the United States are related to bone unification and the reduction of pain and edema in soft tissues.

A number of clinical studies, in vivo animal experiments and in vitro cellular and membrane research suggest that magnetic and electromagnetic field stimulation may accelerate the healing processes. It is now clear that endogenous electromagnetic and magnetic interactions are associated with many basic physiological processes ranging from ion binding and molecular conformation in the cell membrane to macroscopic alterations in tissues.

Since the middle of the 1990s, permanent magnets have become widely used in the US for pain relief. Several recent studies report reduction of pain in post-polio patients (up to 76%), fibromyalgia (up to 32%), peripheral neuropathy (up to 33%), and post surgical wounds (37-65%). These recent studies reported pain management for different etiologies and sites of pain. They demonstrated the potential of a static magnetic field to provide significant pain relief in different disorders.

In a double blind study it was shown that a static magnetic field of 300-500 G significantly decreased the pain score in post polio pain syndrome patients when compared with a placebo group. Another double blind study demonstrated that sleeping on mattresses in which ceramic permanent magnets are embedded (with magnetic field at the target in the range 200-600 G) provided significant benefits to pain and fatigue relief and for sleep in patients suffering from fibromyalgia. The status of the patients in the real treatment group was improved by more than 30%. In a pilot study a significant improvement in 75% of patients with diabetic neuropathy who used permanent magnetic field stimulation on the soles of their feet was found.

Low frequency sine waves and low frequency pulsed electromagnetic fields were used for treatment of pain associated with rotator cuff tendinitis, multiple sclerosis, carpal tunnel syndrome, and periathritis. For example, an improvement has been observed in 93% of patents suffering carpal tunnel pain, and 83% in rotator cuff tendinitis. It has also been reported that 65% of the patients who received daily treatment over 8 weeks for rotator cuff tendinitis were pain-free at the end of the study, as well as 70% of the multiple sclerosis patients who received 15 treatments with low frequency sinewave EMF reported a reduction in spasticity, improvement of bladder control and improvement in endurance.

The PRF modality of EMF therapy has been used for treatment of migraine, chronic pelvic pain, neck pain, and whiplash injuries. In parallel with improvement after the injury, a reduction was reported in the pain of 35% for patients having migraine, accompanied by a significant reduction in occurrence of headaches.

It has become generally accepted that many important systems in living organisms are directed by electromagnetic interactions. This has included not only nerve conductance or cellular membrane structure, but also a whole range of processes that involved ion flow and conformational changes. To date, so far as is known, medicine has utilized electromagnetics mostly for diagnostic purposes. Electrophysiological measurements, electro- and magneto-diagnostics, magnetic resonance imaging, magnetocardiograms and magnetoencephalograms are examples of these diagnostics.

However, electromagnetic fields have still not been widely used in clinical medicine. There are several possible reasons for this fact. First of all, western medicine has been historically based mainly on the achievements of chemistry, which have been further utilized and expanded by the pharmaceutical industry. Unfortunately nearly all pharmaceuticals affect not only the target tissues, but the entire organism and in many cases cause adverse effects in otherwise healthy tissue. Second, medical education has been based upon this chemistry/pharmacology approach, and medical students in the main do not receive significant information about the potential of physical modalities. In contrast with the chemical/pharmacological approach, physical medicine in general, and magnetobiology in particular, have each provided non-invasive, safe and easily applied methods to treat the site of an injury, and the source of pain and inflammation or dysfunction. Magnetotherapy thus has little, if any, side effects. By contrast, in order to reach an appropriate concentration of a pharmacological agent, patients are typically suggested to take dosages which may be several magnitudes larger than needed into the target organ, and this leads to appearance of adverse effects.

The main reasons that MF/EMF are still not widely accepted as treatment modalities in the United States could possibly be the absence of agreement about a common mechanism of action for EMF bioeffects, and an insufficient number of publications about the subject in American medical journals. MF has been recognized as capable of inducing selective changes in the microenvironment around and within the cell, as well as in the cell membrane which in turn may correct selected pathological states.

However, the biophysical mechanism(s) of interaction of weak electric and magnetic fields with biological tissues as well as the biological transductive mechanism(s) remain to be elucidated. The analysis of reported specific reactions to MF/EMF in different biological systems suggests that most of the observed bioeffects strongly depend on the parameters of the applied electromagnetic field.

To study the biophysical mechanisms of MF interactions one should begin with identification of the desired target to MF action. Then the nature of the initial physicochemical interaction of EMF with biological systems, and the expression of these physicochemical changes as a biological response should be investigated. The cell membrane is most often considered the main target for EMF signals. Due to the fact that most of the cellular structures are electrically charged and that the biochemical reactions involve ion transfers it is easy to assume that MF/EMF possess the potential to influence both the structure and function of the most important biochemical/biophysical processes.

Starting from cell size and shape, going through the composition and architecture of the cellular membrane, one can also take into account the different sensitivity of cells based on the above described characteristics. Any change in the electrochemical microenvironment of the cell can cause modifications in the structure of its electrified surface regions by changing the concentration of a specifically bound ion or dipole, which may be accompanied by alterations in the conformation of molecular entities (such as lipids, proteins and enzymes) in the membrane structure. The role of ions as transducers of information in the regulation of cell structure and function is widely accepted. When cells are organized in a tissue, the expected response should include cell-cell communications. In addition, the complexity of the animal/human organism and the existence of compensatory mechanisms, which work on the organism level, must be considered for in vivo experiments.

It is well established that electromagnetic fields (EMF) are capable of eliciting in vivo and in vitro effects from many biological systems. Selected low energy static and time varying magnetic fields have been successfully used to treat therapeutically resistant problems of musculoskeletal system, pain control and cancer. The main advantage of these modalities is that they are non-invasive and may be applied directly to the treatment site.

Often even in the scientific literature magnets and magnetic fields are used as synonyms. One must remember that a magnet itself has no healing capacity. Even the strongest magnets one can find on the market perform their physical action via the magnetic field they generated. Thus, it is the magnetic field, not the piece of material nor the coil, which possesses any healing ability.

The terms “magnetic field” and “electromagnetic fields” refer to different physical phenomena. First, it should be clarified that an electromagnetic field is a combination of a magnetic field and of an electric field. When the amplitude of magnetic field changes, an electric field is generated, and vice versa. Also, any time-varying magnetic field is accompanied with a time-varying electric field.

If a magnetic field is generated via sinusoidal wave generator one should expect the effect to be a result of both magnetic and electric field components. This is even more important in a case when superficial tissues are exposed to magnetic field treatment. While a magnetic field penetrates the biological tissues, the surface of any physical body, including a human body, acts as a barrier for the electric field and the incident electric field has been transferred in an electric current over the body surface.

Another important feature of magnetic/electromagnetic stimulation, especially in the relatively low frequency range, is that electric and magnetic field components behave differently. Once an electric field reaches a material surface, it is transferred into electric current along the surface. Conversely, most materials are transparent to the magnetic field component, which penetrates deep into the body. The depth of penetration depends on the technique of generating the magnetic field. A common problem when assessing the effects initiated by different devices is that each manufacturer uses its own systems and methods of characterizing its product. Many research and clinical trials have been performed without complete dosimetry of the magnetic field at the site of injury and adequate documentation of the exposure conditions.

As a consequence, it is difficult to compare or generalize results obtained at different research or clinical sites. Explanations of experimental protocols even if perfect from a biological or clinical point of view are often incomplete in their characterization of the EMF.

Moreover, there is often confusion among medical practitioners with respect to application of these modalities due to the variety of methods of stimulation, parameters of the applied fields, and lack of a defined biophysical mechanism capable of explaining the observed bioeffects. Animal and human studies have demonstrated that the physical parameters and patterns of application can affect both the type of effect and the efficiency in producing a given response.

In order to achieve good reproducibility of observed biological and clinical effects, one should pay special attention to the detailed dosimetry of the study, use a well-established biological and clinical protocol, and make a complete report of the exposure conditions. Failure to reproduce the reported effects of a biological or clinical study has been, in many cases, due to a failure to explain the exact conditions and/or neglect of some details of the experiments.

For purposes of proper understanding, one must consider that magnetic fields can be differentiated according to the field frequency and amplitude, as well as by other characteristics. The proper term characterizing magnetic flux density (amplitude, field strength) is the Tesla, where 1 Tesla=10,000 Gauss.

The definition of the term “hertz” or the abbreviation “Hz” is well known. The common definition of this term defines a waveform that gradually alternates between a maximum or positive value and an identical minimum but negative value. The number of times this repetitive period is reproduced in one second is the frequency in hertz. In the absence of sinusoidal changes, with any other periodic cycling waveform, the proper term should be “cycles per second”. Therefore, while the term hertz might be useful for familiarity, it should be replaced by “cycles per second” in a number of cases.

If a dosimeter may not be placed inside the volume being subjected to a magnetic field it should be remembered that at least for static and low frequency magnetic fields the magnetic fields measured in air can be regarded as approximately the same as within the human body, assuming the same distance from the source of the field. If the field metrics are not homogeneous throughout the target, care must be taken to determine the variation of the field metrics in order that any replication or further verification by subsequent studies can be deemed reliable and useful.

Investigation of the mechanisms of action of MF on biological systems which are in a state different than their normal physiological one represents the next frontier in electromagnetic biology and medicine. Summarizing the known research data, it is submitted that it is reasonable to state that different MF produce different effects in different biotargets under differing conditions of exposure.

Basic science studies suggest that nearly all participants in the healing process (such as fibrinogen, leukocytes, fibrin, platelets, cytokines, growth factors, fibroblasts, collagen, elastin, keratinocytes, osteoblasts, and free radicals) exhibit alterations in their functions as a result of exposure to MF. Magnetic fields were also shown to affect vasoconstriction and vasodilation, phagocytosis, cell proliferation, formation of the cellular network, epithelization, and scar formation.

Therefore, it is important to evaluate the contribution of MF in the alteration of the basic cellular activities that occur at any one of the distinct stages of tissue repair. The interactions of MF with any structure in the human organism might initiate biophysical and biochemical changes which in turn modify the physiological pathways and accelerate the healing process.

### SUMMARY OF THE INVENTION

The present invention provides a new mechanism of therapeutic action of electromagnetic fields, based upon systemic effect. Briefly, this explains the possibility of selected EMF signals to cause effect at sites distant from the application of the field. One particular embodiment consists of pulsating EMF generated by the means described further for the purpose of EMF therapy. The method of generating specific EMF waveshape and application of this exogenous signal for therapeutic purposes allows the benefit to be achieved via systemic effect. The signal according to the present invention takes the form of a repetitive sequence of semi sinewaves, resulting in a pulsating waveform occurring at a frequency in the range of from about 50 to about 300 pulses per second. In the present embodiment the semi sinewaves are separated by a short DC component, which duration depends on the impedance of the whole system. The signal applied to the patient's limb could be as strong as 4000 Gauss. Simultaneously, reduction of discomfort and pain can be achieved.

The present invention may take the form of methods or apparatus of exogenous therapeutic application of an electromagnetic therapy field to a therapy recipient. It may also be performed in a computer implemented method or computer system, and may be in the form of a computer program product.

### BRIEF DESCRIPTION OF THE DRAWINGS

FIG. 1 is a schematic diagram of a magnetic field generator according to the present invention.

FIG. 1A is an isometric view of a magnetic field generator adapted for use with a therapy recipient's hand and lower arm.

FIG. 1B is an isometric view of a magnetic field generator adapted for use with a therapy recipient's lower leg and foot.

FIG. 2 is a schematic diagram of a cooling system for the magnetic field generator of FIG. 1.

FIG. 3 is a waveform diagram of the waveform of the magnetic field applied as a function of time by the magnetic field generator of FIG. 1 for systemic therapeutic effect according to the present invention.

FIG. 4 is a schematic electrical circuit diagram of the electrical control circuit for forming the waveform of FIG. 3 in the magnetic field generator of FIG. 1.

FIG. 5 is a functional block diagram of processing steps during computerized control of magnetic field generation according to the present invention in the magnetic field generator of FIG. 1.

### DETAILED DESCRIPTION OF THE PREFERRED EMBODIMENTS

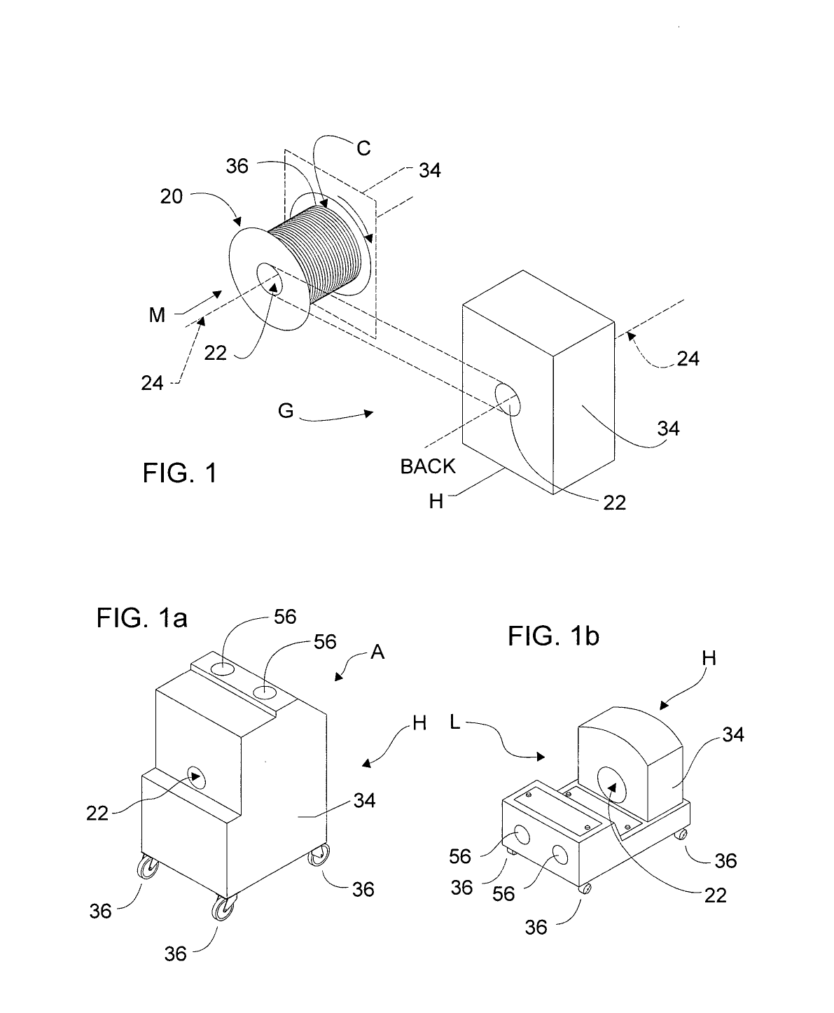

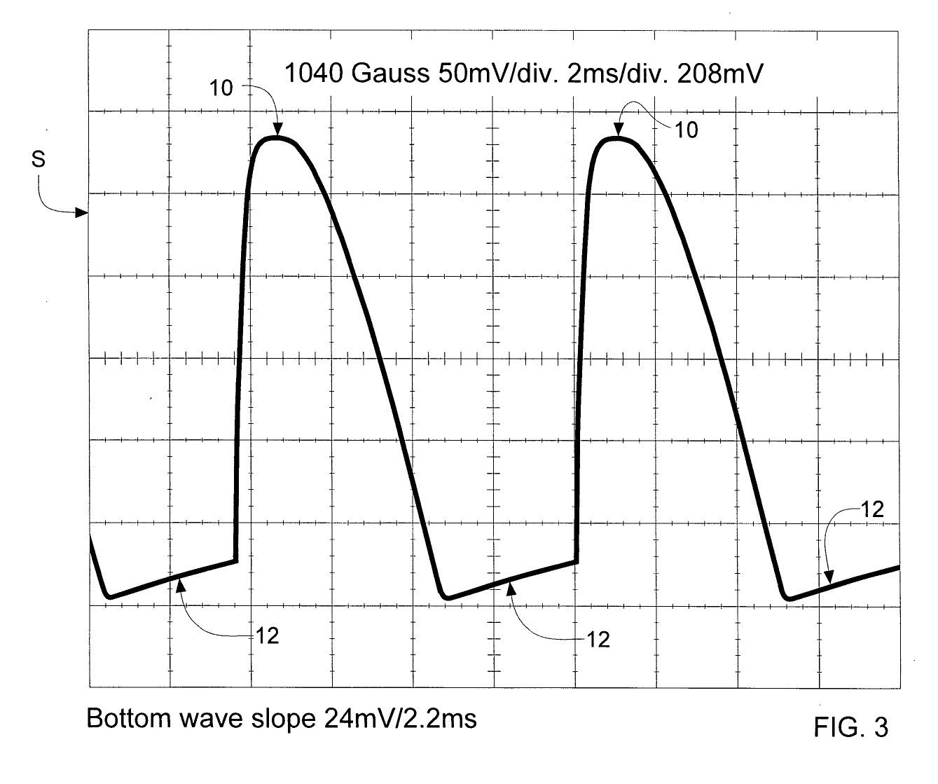

The field generation G is shown in general schematic form in FIG. 1 and may take the specific form of an arm generator A (FIG. 1A) for application of the electromagnetic therapy to the lower arm or hand of a recipient. Alternatively, a generator L (FIG. 1B) may be used for application of electromagnetic therapy to the lower leg, ankle and foot of a therapy recipient. In the drawings (FIG. 3) an electromagnetic field signal S according to the present invention is depicted. With the present invention, the signal waveform S is generated in a magnetic field generator G shown schematically in FIG. 1.

Magnetic fields in the form of the signal S formed by the generator G, A or L may be of a selected range of relatively high field strengths. For example, the arm generator is capable of providing a signal S of strengths of up to about 4,000 Gauss, and the leg generator L is capable of proving a signal S of strength of up to about 1,000 Gauss.

The electromagnetic field having the signal waveform S according to the present invention is applied for therapeutic purposes via upper or lower limbs. The electromagnetic field signal S, (FIG. 3), takes the form of repetitive sequence of semi sinewaves pulses 10 at a frequency in the range of from about 50 to about 300 pulses per second. Further, alternate ones of the repetitive sequence of semi sinewaves are separated by DC components 12 for a time lasting up to 40 percent of the time duration of individual ones of the sequence of semi sinewaves.

The shape of the waveform S which is the subject of the present invention is formed in the generator G (FIG. 1) as a result of bridge rectification of a sinusoidal alternating current wave at the frequency of the power mains, typically at 60 Hz in North America. In other parts of world, 50 Hz is used in the distribution lines and would thus represent the wave which is subject to bridge rectification.

The signal waveform S is a result of the bridge rectification of the alternating current being reversed or flip-flopped in polarity for alternating half-cycles, causing the formation when applied to the coil of a pulsating magnetic field, having 120 pulses per second from 60 Hz main power distribution frequency.

The magnetic field waveform S may be applied as has been described to a limb or limb portion, usually a lower arm portion of a recipient, by the arm unit or generator A (FIG. 1A) to receive electromagnetic field therapy with consequent systemic effect, as will be described. The magnetic field waveform S may be also applied to a lower limb or limb portion, usually a lower leg portion or foot/ankle of a therapy recipient or user, by the leg unit or generator L (FIG. 1B) to receive electromagnetic therapy with consequent systemic effect therapy in a like manner.

In either form, the magnetic field generator G (shown schematically in FIG. 1) is intended for therapeutic applications, generating the electromagnetic field in the form of the waveform S to limbs of a therapy recipient or user. The magnetic field generator G according to the present invention delivers the desired waveshape to at least one coil which permits the magnetic lines of flux to be concentrated in a specific area where a recipient of therapy may place a limb or limb portion to receive therapy. The coils are designed for intermittent application of electromagnetic fields of waveform or signal S for fixed time intervals. The strength of this pulsating magnetic field generated can be varied according to therapeutic needs and can be as high as approximately 4000 Gauss, or 0.40 Tesla.

The magnetic field therapeutic system subject to the present invention includes a magnetic field generating coil C in the form of a wound wire coil. The coil C is located about a generally tubular mounting member M in the form of a spool or bobbin 20. The mounting member M has an opening or passage 22 formed in a longitudinal central portion 24 to allow insertion of a recipient's limb or limb portion. This is typically done by insertion of a limb to be located centrally within the coil C to receive the electromagnetic field generated in the form of signal S by the coil C.

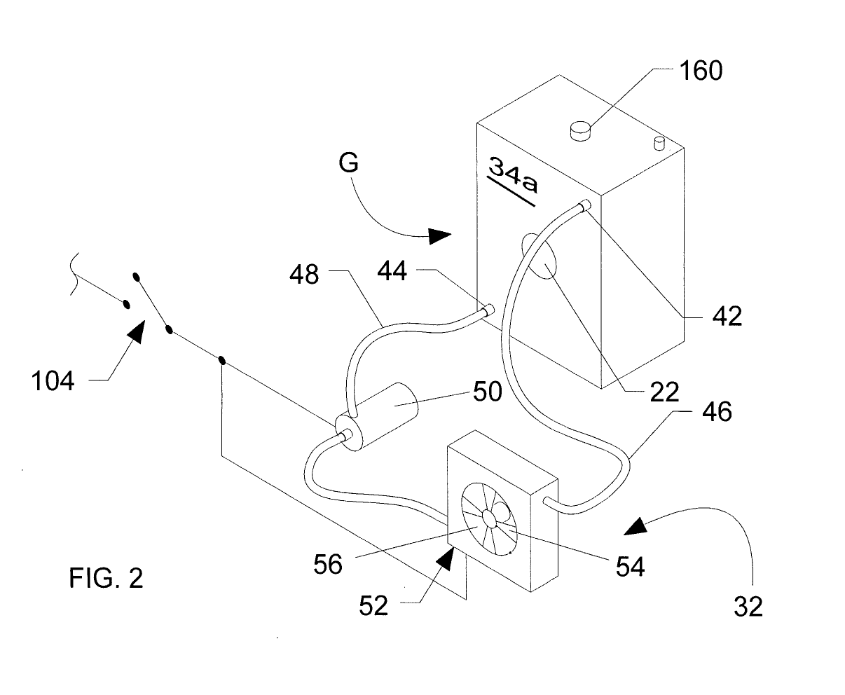

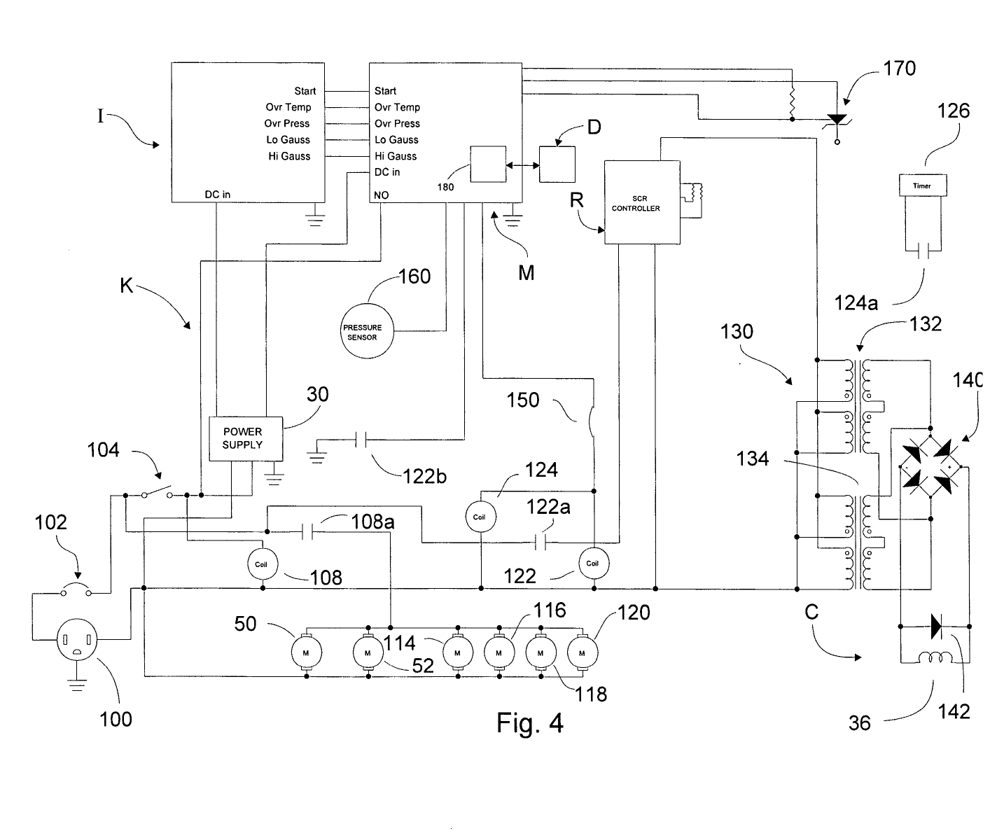

The magnetic field generator G also includes a power supply circuit 30 (FIG. 4) which furnishes current to the coil C and causes the coil to generate the electromagnetic field signal S within the opening 22 of mounting member M for application to the recipient's limb located therein. The magnetic field generator G also includes a control circuit K (FIG. 4) which forms the signal S and regulates the amount of such current flowing to the magnetic coil C to control the intensity of the magnetic field generated. A cooling system 32 (FIG. 2) is also provided to remove excess heat from the magnetic coil C and other portions of the generator G.

The magnetic field generator G is contained within a housing H (FIGS. 1, 1A or 1B) of stainless steel or other suitable material. The housing H includes a cabinet or chamber 34 which contains the power supply circuit 30 and the control circuit K. The size and shape of the cabinet are of appropriate dimensions according to the size of the opening 22 for entry of a recipient's arm (FIG. 1A) or leg (FIG. 1B). The housing H is mounted for movement on wheels 36 (FIGS. 1A and 1B) rotatably mounted beneath lower corner portions of the cabinet 34.

The magnetic coil C may, if desired, be mounted in a cylindrical or toroidal shaped magnetic shield enclosure or cover of the type shown in U.S. Pat. No. 6,541,520 formed of a suitable magnetic shielding material, such as a high permeability steel alloy to substantially confine the electromagnetic lines of flux from the coil C to the general area of the opening 22. The subject matter of that U.S. Patent is incorporated herein by reference for all purposes.

The electromagnetic coil C is preferably of an air core solenoid type, wrapped on the spool or bobbin 20, which is formed of a suitable strength non-magnetic material. Coil windings 36 are enclosed within a container and immersed in a suitable coolant, such as an FR3 oil, to facilitate heat transfer from the magnet coil C. If desired, a pump lubricating oil may be included in the coolant oil. The coolant oil is circulated by a coolant supply system 32 (FIG. 2).

When producing field intensities of strengths of the ranges provided with the present invention, significant heat is generated. The cooling system 32 is provided to remove excess heat from the generator G and to facilitate cooling is a closed loop system which contains a supply of the heat exchange or coolant fluid. The generator G includes an inlet port 42 (FIG. 2) suitably located, such as on a rear surface 34a of the cabinet 34, and an outlet port 44 connected by conduits 46 and 48, respectively, to a pump 50. The coolant fluid is moved through the cooling system 40 by the pump 50 (FIGS. 2 and 4) to the generator G, and also passes S through a heat exchanger 52. The coolant fluid in the heat exchanger 52 is cooled by air moved by a fan 54 at a fan outlet 56. After cooling in the heat exchanger 52, the coolant fluid flows through the conduit 46 to the inlet port 42. Fluid from the generator G is transported by the conduit 48 as a result of the action of pump 50 to the heat exchanger 52.

Considering the electrical control circuit K of the apparatus more in detail, operating electrical power is provided through an electrical connector 100 and fuse 102 through and operating power or on-off switch 104. The power supply circuit 30 (FIG. 4) develops the required adjustable level of DC voltages from conventional AC line 120 voltage input 100 to provide power at levels required by the magnet coil C. The direct current power supply P is furnished incoming power when the switch 104 is closed. The power supply 30 also converts incoming power to suitable direct current operating power levels which are provided to the electronic components of the electrical control circuit K.

A relay coil 108 is connected to the operating power switch 104 and receives electrical current when the switch 104 is in an on position. The relay coil 108 has a set of contracts 108a which close when the coil 108 receives current. The contracts 108a when closed allow operating electrical power to flow to the coolant pump 50, the heat exchanger fan 52, and a suitable number of cooling fans. In the embodiment illustrated, four cooling fans 114, 116, 118 and 120 are present adjacent air outlet ports in the cabinet 34.

A second relay coil 122 is connected to a touch screen display/control input unit I in the control circuit. The input unit I operates in conjunction with a microprocessor M or other suitable computer or programmable control mechanism as will be set forth, for administration of electromagnetic therapy according to the present invention. The input unit I may be, for example, a model OP7100 Smart Screen display of the type available from Z-World, Inc. The processor M may be, for example, a programmable model BL 2020 computer also available from Z-World, Inc.

When an equipment operator designates a therapy time indicator area of a display screen I, the second relay coil 122 receives current. A first set of contacts 122a and a second set of contacts 122b associated with the relay coil 122 close when the coil 122 receives current. The contact set 122a when closed connects an SCR controller switch R to receive electrical power so that the SCR controller switch R may control formation of the waveform S. The SCR controller switch may be, for example, a silicon controlled rectifier, or other control signal sensitive electronic switching device or mechanism of appropriate power rating. A suitable example is of the type available from Phasetronics, Inc. The contact set 122b when closed allows a signal to pass to the processor M indicating that the SCR controller switch R is activated and operating.

A third relay coil 124 is connected to receive electrical current in a like manner to the second relay coil 122 when a time interval has been selected on the touch display input control screen of input unit I. When the relay coil 124 receives electrical current, an associated contact set 124a closes and a suitable time display indicator such as an LCD time display 126 is activated. The time display 126 is located in a position visible to the person receiving therapy so that the therapy recipient and others can be aware of the amount of time that magnetic therapy has been provided in a treatment session.

When the SCR controller R is activated, electrical current is selectively allowed to pass over conductors 128 to a transformer 130 in a manner to be set forth to cause formation of the waveform S. The transformer 130 typically is composed of a set of series connected transformer coils 132 and 134. The transformers 132 and 134 increase or step up the output voltage a suitable amount, such as twice the line voltage to the control circuit, or to about 240 volts, and provide the stepped up voltage to a bridge rectifier 140.

The electromagnetic field generating coil C is connected across the bridge rectifier 140 to form the electromagnetic field in the shape of the waveform S (FIG. 3) under control of the SCR control switch R according to the present invention. A protective diode 142 is connected in parallel with the coil C to prevent magnetic field reversal through the coil C.

The SCR control switch R is configured to selectively control the amplitude of the alternating current signal presented to the bridge rectifier 140 by keeping the amplitude of the alternating current at a selected level consistent with the desired direct current or DC component 12 in the waveform S. Further, the SCR control switch R is selectively adjustable or programmable so that its amplitude limiting operation is operational for an interval of time as indicated at 12 in the waveform S for a time lasting from about 5 to about 40 percent of the time duration of individual ones of the semi sinewaves of the waveform S. The SCR control switch R may be manually programmed by adjustment of the resistance values of an externally connected potentiometer network as shown at 128. It should that other adjustments of impedance values of elements in the control circuit K might be made for the same purpose. As another alternative, the SCR control switch R may be otherwise automatically controlled by settings at appropriate input terminals, or by signals provided from the processor M, if desired.

There are safety features present in the generator G. A thermistor or other form of heat-sensitive switch 150 is physically located on or in close proximity to the coil C. The thermistor 150 is set to open and block flow of electrical current to relay coil 122 once a set temperature limit, such as 140° F. is detected. In such an event, contacts 122a associated with coil 122 open and flow of current through SCR controller R to the transformer 130 and thus to the coil C ceases. Accordingly, when the thermistor 150 senses that an undesirable temperature condition is present in the coil C, flow of the operating current to the coil C is interrupted. Thereafter, the generator G can be restarted only through a normal start-up procedure. In such an event, a restart is allowed only if the thermistor 150 senses that a safe operating temperature is present in the coil C.

A pressure sensor 160 (FIG. 2) is mounted to the housing of the coil C and is electrically connected to the controller/processor M. The pressure sensor 160 detects excess pressure conditions in the generator G. In the event an excessive pressure condition is sensed, an indication signal to that effect is furnished to the process/controller M, which causes the system to shut down. An indication of such an event is provided by the controller/processor M to the touch panel display I, and an appropriate error message is then displayed.

A coolant temperature sensor 170 is mounted to the coil coolant flow tubing near the generator G and is electrically connected to the controller/processor M. The coolant temperature sensor 170 detects excess coolant temperature conditions in the coolant flow tubing 46 and 48. In the event an excessive coil coolant temperature condition is sensed, an indication signal to that effect is furnished to the processor/controller M, which causes the system to shut down. An indication of such an event is provided by the controller/processor m to the touch panel display until I, and an appropriate error message is then displayed.

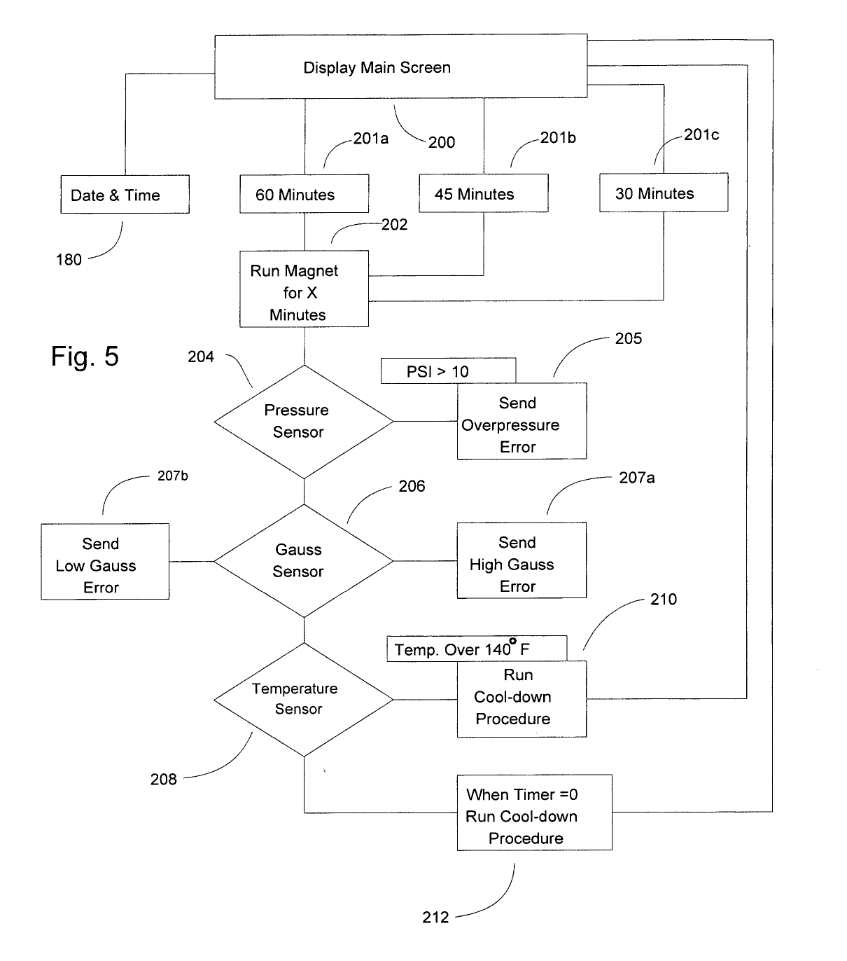

FIG. 5 is a function block diagram of an operation cycle of the touch display unit I and processor M in operation. The unit I and the processor M of the types disclosed above are of the type which are C-programmable, and thus in C programming language. In any case, the processor M receives inputs from the unit I and data or signals form the other components of the control circuit K herein described to undertake the processor logic (FIG. 5) of the present invention, which may be executed by the processor as a series of computer-executable instructions. The instructions may be contained on a data storage device D with a computer readable medium, such as a computer diskette shown in FIG. 4 having a computer usable medium stored thereon. Or, the instructions may be stored in memory 180 of the processor M, or on magnetic tape, conventional hard disk drive, electronic read-only memory, optical storage device, or other appropriate data storage device. FIG. 5 illustrates the structure of the logic of the present invention as embodied in computer program software performable on the processor M, or on a mainframe, personal computer or other form of programmable control device. Those skilled in the art will appreciate that the flow charts illustrate the structures of computer program code elements including logic circuits on an integrated circuit that function according to this invention. Additionally, the flowcharts disclose the functions of the software that would be readily apparent to one of even nominal skill in the art. Furthermore, the flow charts are readily convertible into any of a number of computer program languages based on the type of computer in which the present invention is implemented. Manifestly, the invention is practiced in its essential embodiment by a machine component that renders the program code elements in a form that instructs a digital processing apparatus (that is, a computer) to perform a sequence of function steps corresponding to those shown.

It is important to note that, while the present invention is described in the context of a fully functional digital platform, those skilled in the art will appreciate that the programmable features of the present invention are capable of being distributed as a program product in a variety of forms, and that the present invention applies equally regardless of the particular type of signal-bearing media utilized to actually carry out the distribution. Examples of signal-bearing media include: recordable-type media, such as floppy disks, hard disk drives, and CD ROMs, and transmission-type media such as digital and analog communication links.

A step 200 is the normal wait or standby state during which the display screen unit I indicates to an operator the various functionalities available. When an operator desires an action to be taken, an appropriate area of the touch display screen is contacted. A DATE & TIME display area on the display screen of the unit I, as shown at 180 when activated, indicates the current date and time to be present on the display screen. A suitable number of therapy administration time interval options, such as 60 MINUTES, 45 MINUTES and 30 MINUTES as shown at 201a, 201b and 201c, are present on the touch display screen of input unit I to allow a therapy administrator or operator to activate the coil C for a corresponding amount of time as shown at step 202. With the present invention, the therapy administration time interval is set or established so that the portion of the therapy recipient's anatomy, usually an arm or lower leg, is maintained in the magnetic field of the coil C in the form generated by the waveform S for a time interval adequate to insure that the full volume of blood present in the recipient's body is subjected to the generated magnetic field. The therapy administration time interval so established for the time of operation of the coil C depends on the body size and body mass of the therapy recipient, but in most cases is on the order of about sixty minutes. The therapy applied by the coil C of the generator G is thus exogenous, in that the field is generated and applied to the therapy externally and without physical contact with the recipient's body. Further, the exogenous electromagnetic field is applied for a time adequate to allow the full volume of blood in the recipient's body to be subject to the therapeutic effects of the electromagnetic field. Because of the time of exposure of the therapy recipient to the electromagnetic field of the coil C in the generator G, a systemic effect of therapeutic application is achieved. Further explanation of the systemic effect provided with the present invention is set forth below.

During the time of operation of the coil C, the pressure sensor 160 is monitored, as indicated at step 204, and if excess pressure is sensed, an OVERPRESSURE error is provided as indicated at step 205 by processor M to touch panel display I. Similarly, the magnetic field strength is monitored by a gauss meter or other field strength measuring devise, as indicated at step 206, and if the field strength of the coil C is sensed to be unsatisfactorily high or low, a corresponding high or low gauss error as indicated at 207a and 207b is provided to touch panel display unit I. In a like manner, the condition of coil housing temperature sensor thermistor 150 is monitored, as indicated at step 208, and if the thermistor 150 has opened because an excess coil temperature is present, an equipment cool-down cycle is begun as shown at 210 and appropriate error is provided to touch panel display unit I.

Normally, in the absence of unsatisfactory equipment operation conditions being detected by the safety features, the coil C of unit I furnishes electromagnetic therapy in the waveform S for the time set on the display screen until the established therapy time has elapsed as shown at 212. At such a time, the coil C is deactivated by the processor M, and the normal cool-down procedure occurs, with the touch display unit I and controller processor M returning to the normal standby step 200.

Magnetotherapy provides non-invasive, safe and easy to apply methods to treat a large variety of injury, pathology, pain, inflammation and other types of dysfunction.

The present invention consists of application of magnetic or electromagnetic fields for therapeutic purposes by way of systemic effects for the treatment of various injuries and diseases. Prior to the present invention, there has been a traditional approach to magnetic field therapy based upon target oriented magnetic field application. The applicator has been placed over the targeted tissue/organ, and the generated field adjusted in a way that the target received a desired magnetic flux density. This allowed the target to receive a known and predictable dosage, while in the case of any pharmacotherapy the dosage was selected mainly by the weight of the patient.

As a result of the inherent interactions between organs, tissues, and cells, one should point out, that at the core of observed sensitivity to low-level EMF are a series of cooperative processes. Therefore it appears reasonable to consider the fact that even if the target organ received 100% of a delivered dose of applied EMF, the cellular communications, signal transductions and intersystem interactions might cause effects at some other system, organ, or tissue. This resulted in alterations in the functioning of these systems, organs, or tissues including healing. The alterations were found to be greater when the disease or injury was more severe, usually the alterations were still in the possible compensatory range. Therefore, an appropriate choice of magnetic and/or electromagnetic field may be expected to initiate systemic changes that result in efficacious effects distant from the point of application. This phenomenon may appear in at least two forms: (1) neutralization of the pain experience distant to the point of magnetic field exposure; and, (2) various alterations of T-lymphocytes in response to pain and to magnetic fields.

The present invention is focused on understanding the principle that the functions of most, if not all, systems in human body are based upon electric effects. For example, the blood flow is actually movement of charged particles; thus, any transport phenomena depends on electric charges and potentials.

The appropriate choice of magnetic/electromagnetic fields can initiate changes at the systemic level, which further may be manifested at locations distant to the place of therapy application. It is even more important in conditions such as pain control, when the manifestation of the pain may not be obligatory in the place pain occurred. In that situation systemic effect in reducing inflammation should be connected with both blood vessels and lymphatic systems, and the contents there in.

The underlying principle in consideration of systemic effects is that in this manner the exploration of living systems from the subcellular level to the level of organism including inquiry into the movement of charges along biochemical pathways and along vascular channels can be achieved.

It is known that each cell in an organism has a surface charge (mostly negative). Furthermore, the blood flow may be visualized as a movement of charged particles within larger or smaller blood vessels. For that reason, during normal functioning of an organism, blood components, such as erythrocyte, lymphocytes, leukocytes, and other formed elements are traveling within weak electromagnetic fields created by surrounding tissues, including blood vessels themselves.

The blood plasma and interstitial fluids are examples of ionic media capable of effectively conducting current. Blood vessels and interstitial fluids effectively serve in the same manner as electrical cables that carry currents transporting charged particles over short and long distances.

There is no single cell in human body which is not involved one way or another in system interactions. The text-book examples include the blood-vessels and lymphatic system, and the central nervous system; however, independently or in synchronization with the above mentioned systems, other systems such as the endocrine, immune, and respiratory systems are working and interact with each other.

Another feature of the present invention consists in the release of certain molecules that have effects locally as well as systemically (i.e., at points distant from the area of application). Locally, these molecules may initiate a cascade of intracellular events leading to improved cell function; and, some of these molecules will enter the circulation and elicit their efficacious effects systemically on cells, tissues, and organs providing a basis for some of the observed therapeutic effects. This more likely is complimented by the fact that in the human body the organs and tissues are coordinated in a functionally complete system in which each organ/tissue acts in concert with all other organs and systems.

It is known in the art that gradients in the electrical potentials exist between organs throughout the body and are related to the metabolic activity of the cells, tissues, and organs. The question arises: What happens in case of injury or disease? Conventional wisdom suggests that these potentials and their gradients should change with any change in metabolic activity. Once the potential gradients are changed, the currents flowing through the cells, tissues, and organs of that region are altered. Again, the systemic effect of the exogenous magnetic field from our invention will lead to the necessary corrections.

With the present invention, it has been found that externally applied magnetic fields can reduce pain, enhance healing, and efficaciously affect the organism; and, no doubt, this is influenced by, not only charges and ions moving in the blood stream but also the vascular beds within the organs. Additionally, magnetic fields can directly influence the state and flow of interstitial liquid, and may indirectly influence the appearance of weak electrical interactions between blood plasma, blood vessels, interstitial fluid, and cells.

Taken together, the cooperativity, signal transportation and transduction can not exist without the enormous potential of living matter to perform signal amplification, thereby making possible appearance of effects at sites distant from stimulation site.

It is known in the art that magnetic fields influence signal perception by the cells, tissues, organs, and systems; thus, the signal amplification and transduction could modify the whole range of biochemical processes. It is an even more important possibility for the organs and tissues that are out of balance due to diseases or injuries. Magnetic fields could help in restoration of normal functioning of injured tissues.

With the present invention it has been found that magnetic fields reduce pain, presumably by evacuating the excessive accumulation of interstitial fluid which results in an increase of tissue pressure, leading to activation of nerve endings. The same mechanism is likely for relaxation after heavy physical work or exercising.

Thus, it seems highly likely that an exogenous electromagnetic field is, with the waveform S, capable of initiating changes in the moving fluids in vessels and in the interstitial space therefore making possible restoration of normal functioning and healing.

The blood circulation is often considered as a nonselective mechanical transportation for oxygen, ions, hormones, numerous neuroendocrine secretions, chemical and various other substances that are delivered to the tissues by diffusion, filtration and osmosis through capillaries. However, when exposed to electromagnetic fields, theses substances become transporters of energy and information and their delivery to the target tissues allocated at distance from the exposure site will first be changed. Second, the substances will initiate responses at the target tissues different from the normal delivery to tissues. When the tissue is diseased or injured, these effects become more important, because following a correct selection of the magnetic field conditions, they would significantly enhance the healing.

The capacitive or inductive coupling of MF/EMF to the level of systemic effects makes this approach suitable for treatment of injury and disease, including malignancy. This approach relies on the interactions between externally applied fields with charges and dipoles within the cells, tissues, and organs. Thus, the notion of single cells exposed to EMF should be substituted with the effects seen on more complicated level as tissues, organs, and systems.

Since in malignancy, a difference in electrical potential between tumor tissue and surrounding normal tissues arises, the healing should also include the elimination of this potential difference, and restoration of normal metabolism, including normal electrical properties. This balancing process can be accelerated by appropriate electric/electromagnetic simulation.

The nervous system relays information by direct contact as adrenergic or cholinergic molecules are released at nerve ending and synapses. Hormones and neurotransmitters are carried via circulation systems to specific receptors at sites distant from their point of origin. Much less is known about the immune system, although it is clear that its communications include both humoral and neural connections. In the final analysis all the messages are transmitted by means of weak energy transfer across cellular membranes. The word “message” is used because the signal transduction cascade involves not only transport of material and energy, but transport of information, as well.

Having in mind that even weak magnetic fields are capable of changing the microstructure of water cluster or of the hydration layers, one may consider that external electromagnetic field might initiate such changes. It appears reasonable to link the potential for pain relief to the possibility to alter the water structure. Not the water as individual molecules, but the water involved in hydration of ions, proteins, cellular structures, cytoplasm and intercellular spaces.

With the present invention, it is felt that when a blood vessel is exposed to an electromagnetic field, the changes in water structure, in the hydration of charged erythrocytes, lymphocytes, leukocytes, and molecules could be “memorized” and transferred to tissues/organs distant from the site of interaction. Thus the changes in the physical properties of cellular and tissue water might reduce the degree of chronic pain experienced by a human.

It is known that for Jurkat cells the effect of exposure to magnetic fields is stronger when T-lymphocytes were fully activated, and basically absent in non-activated cells. If this observation is extrapolated to the system level, the interpretation leads to a conclusion that a “healthy” cell neglects the applied field, while cells activated by some reason like inflammation cells react in a detectable manner.

Having in mind that the T-lymphocytes are one to the key players in the controlling of inflammation, the state and performance of the T-lymphocytes become plausible criteria for evaluation of the systemic effect of EMF.

Lymphocytes are recognized as a part of the defense mechanism of the human body. The presence is a primary mechanism to encapsulate and destroy the abnormal cancer cells. If this hypothesis is correct, then sophisticated analysis of the dynamic properties and types of lymphocytes may offer a simple, reliable and conclusive prognostic test for the potential of using EMF to fight cancer.

According to the present invention, it is submitted that these interactions influence not only one target organelle or molecule, but also reflects on the entire system this target is serving with. It is also submitted that even within one system the effect might be seen at certain distance from the site of EMF application. Furthermore, interactions of different systems within a human organism by different mechanisms and pathways may cause alterations in a system that is not directly involved in these interactions.

The response of a system out of equilibrium (in case of disease and/or injury) is stronger than of a system in normal status. It should be also considered that once every single component of a system is out of balance, by the negative feedback mechanisms the whole system undergoes small or large perturbations.

Test data to date has shown that selected magnetic fields generated by the waveforms (120 pps, up to 1,500 Gauss) when applied to an uninvolved limb are capable of initiating effects at sites distant from application. This has been observed in many conditions such as, but not limited to, Parkinson's disease, peripheral neuropathy, and reflex sympathetic dystrophy (RSD) (also referred to as chronic regional pain syndrome-CRPS).

The foregoing disclosure and description of the invention are illustrative and explanatory thereof and various changes in materials, sizes, and configurations, as well as methods of use and the particular electromagnetic field therapy may be made without departing from the spirit of the invention, the scope of which is defined in the following claims.

While the invention has been described herein with respect to certain embodiments, it should be understood by those that are skilled in the art that is not so limited. The invention is susceptible of various modifications and changes without departing from the scope of the claims.

What is claimed is:

1. An electromagnetic field activation signal for generating an electromagnetic therapy field for exogenous therapeutic application by an electromagnetic coil for treatment by systemic effect of a therapy recipient by application of the therapy field to the recipient at a location on the recipient's body different than the area of the recipient's body requiring therapy, the electromagnetic field activation signal comprising:
a repetitive sequence of semi sinewaves at a frequency in the range of from about 50 to about 300 pulses per second; and
alternate ones of the repetitive sequence of semi sinewaves being limited in amplitude for a time lasting from about 5 to about 40 percent of the time duration of an individual one of the semi sine waves.

2. The electromagnetic field activation signal of claim 1, wherein the electromagnetic therapy field has a strength of up to 4,000 Gauss.

3. The electromagnetic field activation signal of claim 1, wherein the electromagnetic therapy field has a strength of up to 1,500 Gauss.

4. The electromagnetic field activation signal of claim 1, wherein the electromagnetic therapy field has a strength of up to 1,000 Gauss.

5. The electromagnetic field activation signal of claim 1, wherein the semi sinewaves occur at a frequency of from about 50 to about 100 pulses per second.

6. The electromagnetic field activation signal of claim 1, wherein the semi sinewaves occur at a frequency of from about 60 to about 120 pulses per second.

7. The electromagnetic field activation signal of claim 1, wherein the therapy field is for application to a therapy recipient for therapeutic treatment of Parkinson's disease.

8. The electromagnetic field activation signal of claim 1, wherein the therapy field is for application to a therapy recipient for therapeutic treatment of peripheral neuropathy.

9. The electromagnetic field activation signal of claim 1, wherein the therapy field is for application to a therapy recipient for therapeutic treatment of reflex sympathetic dystrophy.

10. The electromagnetic field activation signal of claim 1, wherein the therapy field is for application to a therapy recipient for therapeutic treatment of an injury selected from the group consisting of reduction of edema, contusions and connective tissue tears.

11. The electromagnetic field activation signal of claim 1, wherein the therapy field is for application to a therapy recipient for therapeutic treatment of an injury selected from the group consisting of post-surgical recovery, wound healing and severe burns recovery.

12. The electromagnetic field activation signal of claim 1, wherein the therapy field is for application to a therapy recipient as adjuvant therapy in connection with chemotherapy.

13. The electromagnetic field activation signal of claim 1, wherein the therapy field is for application to a therapy recipient as adjuvant therapy in connection with radiation therapy.

14. The electromagnetic field activation signal of claim 1, wherein the therapy field is for application to a therapy recipient for therapeutic treatment of cancer.

15. The electromagnetic field activation signal of claim 1, wherein the therapy field is for application to a therapy recipient for therapeutic treatment of carpal tunnel syndrome.

16. The electromagnetic field activation signal of claim 1, wherein the amplitude limited alternate ones of the repetitive sequence of semi sinewaves are limited in amplitude to from about 20 to about 40 percent of the peak amplitude of the semi sinewaves.

17. An electromagnetic field activation signal for generating an electromagnetic therapy field for exogenous therapeutic application by an electromagnetic coil to a therapy recipient for treatment purposes, the electromagnetic field activation signal comprising:
a repetitive sequence of semi sinewaves at a frequency in the range of from about 50 to about 300 pulses per second; and
alternate ones of the repetitive sequence of semi sinewaves being limited in amplitude for a time lasting from about 5 to about 40 percent of the time duration of alternate ones of the semi sine waves.

18. An apparatus for exogenous therapeutic application of an electromagnetic therapy field to a therapy recipient for systemic effect purposes, comprising:
a housing having an opening and a passage therein for insertion of a limb of a therapy recipient;
an electromagnetic field generating coil located around the passage in the housing; and
a control circuit forming a control signal to generate an electromagnetic field signal forming the electromagnetic therapy field for exogenous therapeutic application by the electromagnetic coil to the therapy recipient for therapeutic purposes at a location on the recipient's body different than the limb inserted in the passage in the housing, the electromagnetic field activation signal comprising a repetitive sequence of semi sinewaves at a frequency in the range of from about 50 to about 300 pulses per second, and having alternate ones of the repetitive sequence of semi sinewaves being limited in amplitude for a time lasting from about 5 to about 40 percent of the time duration of the semi sinewaves.

19. The apparatus of claim 18, wherein the passage in the housing is configured for insertion of a lower portion of an arm of the therapy recipient.

20. The apparatus of claim 19, wherein the passage in the housing is configured for insertion of a hand of the therapy recipient.

21. The apparatus of claim 19, wherein the passage in the housing is configured for insertion of a wrist of the therapy recipient.

22. The apparatus of claim 19, wherein the passage in the housing is configured for insertion of a forearm of the therapy recipient.

23. The apparatus of claim 18, wherein the passage in the housing is configured for insertion of a lower portion of a leg of the therapy recipient.

24. The apparatus of claim 23, wherein the passage in the housing is configured for insertion of a foot of the therapy recipient.

25. The apparatus of claim 23, wherein the passage in the housing is configured for insertion of an ankle of the therapy recipient.

26. The apparatus of claim 23, wherein the passage in the housing is configured for insertion of a foreleg of the therapy recipient.

27. The apparatus of claim 18, wherein the electromagnetic therapy field formed by the electromagnetic field generating coil has a strength of up to 4,000 Gauss.

28. The apparatus of claim 18, wherein the electromagnetic therapy field formed by the electromagnetic field generating coil has a strength of up to 1,500 Gauss.

29. The apparatus of claim 18, wherein the electromagnetic therapy field formed by the electromagnetic field generating coil has a strength of up to 1,000 Gauss.

30. The apparatus of claim 18, wherein the semi sinewaves formed by the electromagnetic field generating coil occur at a frequency of from about 50 to about 100 pulses per second.

31. The apparatus of claim 18, wherein the semi sinewaves formed by the electromagnetic field generating coil occur at a frequency of from about 60 to about 120 pulses per second.

32. The apparatus of claim 18, wherein the amplitude limited alternate ones of the repetitive sequence of semi sinewaves formed by the electromagnetic field generating coil are limited in amplitude to from about 20 to about 40 percent of the peak amplitude of the semi sinewaves.

33. The apparatus of claim 18, wherein the control circuit includes a bridge rectifier.

34. The apparatus of claim 18, wherein the electromagnetic field generating coil a plurality of series connected electromagnetic field generating coils.

35. The apparatus of claim 34, further a diode connected to the electromagnetic field generating coil to prevent field reversal therein.

36. The apparatus of claim 18, wherein the control circuit includes an electronic switch to limit the amount of current flow to the electromagnetic field generating coil for the duration of the limited amplitude of the alternate ones of the repetitive sequence of semi sinewaves.

37. The apparatus of claim 36, wherein the electronic switch comprises a silicon controlled rectifier.

38. The apparatus of claim 36, wherein the control circuit includes a processor controlling the operation of the electronic switch.

39. The apparatus of claim 36, further including a display unit for indicating the status of application of electromagnetic therapy by the electromagnetic field generating coil.

40. The apparatus of claim 39, wherein the display unit includes an input mechanism for receiving electromagnetic therapy operation controls.

41. The apparatus of claim 36, wherein the operating cycle of electronic switch is adjustable.

42. The apparatus of claim 18, further including a cooling system for cooling the electromagnetic field generating coil.

43. The apparatus of claim 18, further including a sensor for sensing temperature in the housing.

44. The apparatus of claim 43, wherein the control circuit includes a processor controlling the operation of the electronic switch and the temperature sensor sends a signal to the processor in the event of excessive temperature in the housing.

45. The apparatus of claim 44, wherein the processor terminates operation of the electromagnetic field generating coil in the event of excessive temperature in the housing.

46. An apparatus for exogenous therapeutic application of an electromagnetic therapy field to a therapy recipient for therapeutic purposes, comprising:
a housing having an opening and a passage therein for insertion of a limb of a therapy recipient;
an electromagnetic field generating coil located around the passage in the housing; and
a control circuit forming a control signal to generate an electromagnetic field signal forming the electromagnetic therapy field for exogenous therapeutic application by the electromagnetic coil to the therapy recipient for therapeutic purposes, the electromagnetic field activation signal comprising a repetitive sequence of semi sinewaves at a frequency in the range of from about 50 to about 300 pulses per second, and having alternate ones of the repetitive sequence of semi sinewaves being limited in amplitude for a time lasting from about 5 to about 40 percent of the time duration of the semi sinewaves.

47. A method of forming an electromagnetic field activation signal for generating an electromagnetic therapy field for exogenous therapeutic application by an electromagnetic coil for treatment by systemic effect of a therapy recipient by application of the therapy field to the recipient at a location on the recipient's body different than the area of the recipient's body requiring therapy, comprising the steps of:
receiving an alternating current input signal;
full wave rectifying the received alternating current input signal to form a repetitive sequence of semi sinewaves at a frequency in the range of from about 50 to about 300 pulses per second;
limiting the amplitude of alternate ones of the repetitive sequence of semi sinewaves for a time lasting from about 5 to about 40 percent of the time duration of the semi sinewaves to provide a control signal; and
furnishing the control signal to the electromagnetic coil to form the electromagnetic field activation signal for generating the electromagnetic therapy field.

48. The method of claim 47, wherein the generated electromagnetic therapy field has a strength of up to 4,000 Gauss.

49. The method of claim 47, wherein the generated electromagnetic therapy field has a strength of up to 1,500 Gauss.

50. The method of claim 47, wherein the generated electromagnetic therapy field has a strength of up to 1,000 Gauss.

51. The method of claim 47, wherein the generated electromagnetic therapy field semi sinewaves occur at a frequency of from about 50 to about 100 pulses per second.

52. The method of claim 47, wherein the generated electromagnetic therapy field semi sinewaves occur at a frequency of from about 60 to about 120 pulses per second.

53. The method of claim 47, wherein the generated electromagnetic therapy field is for application to a therapy recipient for therapeutic treatment of Parkinson's disease.

54. The method of claim 47, wherein the generated electromagnetic field is for application to a therapy recipient for therapeutic treatment of peripheral neuropathy.

55. The method of claim 47, wherein the generated electromagnetic therapy field is for application to a therapy recipient for therapeutic treatment of reflex sympathetic dystrophy.

56. The method of claim 47 wherein the generated electromagnetic therapy field is for application to a therapy recipient for therapeutic treatment of an injury selected from the group consisting of reduction of edema, contusions and connective tissue tears.

57. The method of claim 47, wherein the generated electromagnetic therapy field is for application to a therapy recipient for therapeutic treatment of an injury selected from the group consisting of post-surgical recovery, wound healing and severe burns recovery.

58. The method of claim 47, wherein the generated electromagnetic therapy field is for application to a therapy recipient as adjuvant therapy in connection with chemotherapy.

59. The method of claim 47, wherein the generated electromagnetic therapy field is for application to a therapy recipient as adjuvant therapy in connection with radiation therapy.

60. The method of claim 47, wherein the generated electromagnetic therapy field is for application to a therapy recipient for therapeutic treatment of cancer.

61. The method of claim 47, wherein the generated electromagnetic therapy field is for application to a therapy recipient for therapeutic treatment of carpal tunnel syndrome.

62. The method of claim 47, wherein the step of limiting the amplitude of alternate ones of the repetitive sequence of semi sinewaves comprises the step of limiting the amplitude to from about 20 to about 40 percent of the peak amplitude of the semi sinewaves.

63. A method of forming an electromagnetic field activation signal for generating an electromagnetic therapy field for exogenous therapeutic application by an electromagnetic coil to a therapy recipient for therapeutic purposes, comprising the steps of:
receiving an alternating current input signal;
full wave rectifying the received alternating current input signal to form a repetitive sequence of semi sinewaves at a frequency in the range of from about 50 to about 300 pulses per second;
limiting the amplitude of alternate ones of the repetitive sequence of semi sinewaves for a time lasting from about 5 to about 40 percent of the time duration of the semi sinewaves to provide a control signal; and
furnishing the control signal to the electromagnetic coil to form the electromagnetic field activation signal for generating the electromagnetic therapy field.

64. A method of forming an electromagnetic field activation signal for generating an electromagnetic therapy field for exogenous therapeutic application by an electromagnetic coil for treatment by systemic effect of a therapy recipient by application of the therapy field to the recipient at a location on the recipient's body different than the area of the recipient's body requiring therapy, comprising the steps of:
generating an electromagnetic therapy field in a housing containing an electromagnetic coil therein, the electromagnetic therapy field having the form of a repetitive sequence of semi sinewaves at a frequency in the range of from about 50 to about 300 pulses per second, and ones of the repetitive sequence of semi sinewaves being limited in amplitude for a time lasting from about 5 to about 40 percent of the time duration of the semi sinewaves;
inserting a limb of the therapy recipient into the housing; and
subjecting the limb to the generated electromagnetic therapy field for a time sufficient for the full volume of blood flowing in the therapy recipient's body to be exposed to the electromagnetic field therapy.

65. The method of claim 64, wherein the step of inserting comprises the step of inserting a lower portion of an arm of the therapy recipient.

66. The method of claim 65, wherein the step of inserting comprises the step of inserting a hand of the therapy recipient.

67. The method of claim 65, wherein the step of inserting comprises the step of inserting a wrist of the therapy recipient.

68. The method of claim 65, wherein the step of inserting comprises the step of inserting a forearm of the therapy recipient.

69. The method of claim 65, wherein the wherein the step of inserting comprises the step of inserting a lower portion of a leg of the therapy recipient.

70. The method of claim 69, wherein the step of inserting comprises the step of inserting a foot of the therapy recipient.

71. The method of claim 69, wherein the step of inserting comprises the step of inserting an ankle of the therapy recipient.

72. The method of claim 69, wherein the step of inserting comprises the step of inserting a foreleg of the therapy recipient.

73. The method of claim 64, wherein generated electromagnetic therapy field has a strength of up to 4,000 Gauss.

74. The method of claim 64, wherein the generated electromagnetic therapy field has a strength of up to 1,500 Gauss.

75. The method of claim 64, wherein the generated electromagnetic therapy field has a strength of up to 1,000 Gauss.

76. The method of claim 64, wherein the generated electromagnetic therapy field semi sinewaves occur at a frequency of from about 50 to about 100 pulses per second.

77. The method of claim 64, wherein the generated electromagnetic therapy field semi sinewaves occur at a frequency of from about 60 to about 120 pulses per second.

78. The method of claim 64, wherein the generated electromagnetic therapy field is for application to a therapy recipient for therapeutic treatment of Parkinson's disease.

79. The method of claim 64, wherein the generated electromagnetic field is for application to a therapy recipient for therapeutic treatment of peripheral neuropathy.

80. The method of claim 64, wherein the generated electromagnetic therapy field is for application to a therapy recipient for therapeutic treatment of reflex sympathetic dystrophy.

81. The method of claim 64, wherein the generated electromagnetic therapy field is for application to a therapy recipient for therapeutic treatment of an injury selected from the group consisting of reduction of edema, contusions and connective tissue tears.

82. The method of claim 64, wherein the generated electromagnetic therapy field is for application to a therapy recipient for therapeutic treatment of an injury selected from the group consisting of post-surgical recovery, wound healing and severe burns recovery.

83. The method of claim 64, wherein the generated electromagnetic therapy field is for application to a therapy recipient as adjuvant therapy in connection with chemotherapy.

84. The method of claim 64, wherein the generated electromagnetic therapy field is for application to a therapy recipient as adjuvant therapy in connection with radiation therapy.

85. The method of claim 64, wherein the generated electromagnetic therapy field is for application to a therapy recipient for therapeutic treatment of cancer.

86. The method of claim 64, wherein the generated electromagnetic therapy field is for application to a therapy recipient for therapeutic treatment of carpal tunnel syndrome.

87. The method of claim 64, wherein the step of limiting the amplitude of alternate ones of the repetitive sequence of semi sinewaves comprises the step of limiting the amplitude to from about 20 to about 40 percent of the peak amplitude of the semi sinewaves.

88. A method of forming an electromagnetic field activation signal for generating an electromagnetic therapy field for exogenous therapeutic application by an electromagnetic coil to a therapy recipient for therapeutic purposes, comprising the steps of:
generating an electromagnetic therapy field in a housing containing an electromagnetic coil therein, the electromagnetic therapy field having the form of a repetitive sequence of semi sinewaves at a frequency in the range of from about 50 to about 300 pulses per second, and ones of the repetitive sequence of semi sinewaves being limited in amplitude for a time lasting from about 5 to about 40 percent of the time duration of the semi sinewaves;
inserting a limb of the therapy recipient into the housing; and
subjecting the limb to the generated electromagnetic therapy field for a time sufficient for the full volume of blood flowing in the therapy recipient's body to be exposed to the electromagnetic field therapy.

89. A computer-implemented method of exogenous therapeutic application by an electromagnetic coil for treatment by systemic effect of a therapy recipient by application of the therapy field to the recipient at a location on the recipient's body different than the area of the recipient's body requiring therapy, comprising the steps of:
establishing in the computer a set of selected therapy administration times;
responding to selection of one of the set of selected therapy administration times by generating an electromagnetic therapy field in a housing containing an electromagnetic coil therein, the electromagnetic therapy field having the form of a repetitive sequence of semi sinewaves at a frequency in the range of from about 50 to about 300 pulses per second, alternate ones of the repetitive sequence of semi sinewaves being limited in amplitude for a time lasting from about 5 to about 40 percent of the time duration of the semi sinewaves; and
applying the generated electromagnetic therapy field to a limb of the therapy recipient for the selected one of the set of selected therapy administration times.

90. The computer-implemented method of claim 89, further including the step of:
sensing pressure in the housing.

91. The computer-implemented method of claim 90, further including the step of:
terminating the step of generating an electromagnetic therapy field in the event of an excess pressure being sensed.

92. The computer-implemented method of claim 89, further including the step of:
sensing temperature of the electromagnetic coil.

93. The computer-implemented method of claim 90, further including the step of:
terminating the step of generating an electromagnetic therapy field in the event of an excess temperature being sensed.

94. The computer-implemented method of claim 89, further including the step of:
sensing the strength of the electromagnetic therapy field being generated.

95. The computer-implemented method of claim 92, further including the step of:
adjusting the strength of the electromagnetic therapy field being generated in the event an unacceptable strength level is sensed.

96. A computer-based system for exogenous therapeutic application by an electromagnetic coil for treatment by systemic effect of a therapy recipient by application of the therapy field to the recipient at a location on the recipient's body different than the area of the recipient's body requiring therapy, the computer-based system comprising:
a housing having an opening and a passage therein for insertion of a limb of a therapy recipient;
an electromagnetic field generating coil located around the passage in the housing;
a processor for performing the steps of:
establishing in the computer a set of selected therapy administration times;
responding to selection of one of the set of selected therapy administration times by generating an electromagnetic therapy field in the electromagnetic coil, the electromagnetic therapy field having the form of a repetitive sequence of semi sinewaves at a frequency in the range of from about 50 to about 300 pulses per second, alternate ones of the repetitive sequence of semi sinewaves being limited in amplitude for a time lasting from about 5 to about 40 percent of the time duration of the semi sinewaves; and
applying the generated electromagnetic therapy field to a limb of the therapy recipient for the selected one of the set of selected therapy administration times.

97. A computer program product stored in signal bearing media for causing a processor to control exogenous therapeutic application by an electromagnetic coil for treatment by systemic effect of a therapy recipient by application of the therapy field to the recipient at a location on the recipient's body different than the area of the recipient's body requiring therapy, the computer program product causing the processor to perform the steps of:
establishing in the computer a set of selected therapy administration times;
responding to selection of one of the set of selected therapy administration times by generating an electromagnetic therapy field in the electromagnetic coil, the electromagnetic therapy field having the form of a repetitive sequence of semi sinewaves at a frequency in the range of from about 50 to about 300 pulses per second, alternate ones of the repetitive sequence of semi sinewaves being limited in amplitude for a time lasting from about 5 to about 40 percent of the time duration of the semi sinewaves; and
applying the generated electromagnetic therapy field to a limb of the therapy recipient for the selected one of the set of selected therapy administration times.
